(19) United States
(12) Patent Application Publication
(10) Pub. No.: US 2026/0345855 A1
(43) Pub. Date: Nov. 5, 2026

(54) WASTE CONTROL UNIT AND WASTE CONTAINER COMPRISING A WASTE CONTROL UNIT

(71) Applicant: OI SOURCING AB, Falsterbo (SE)

(72) Inventor: Jan Arlemark, Malmö (SE)

(21) Appl. No.: 19/111,840

(22) PCT Filed: Aug. 25, 2023
(86) PCT No.: PCT/IB2023/058437

§ 371 Date: Mar. 14, 2025

(30) Foreign Application Priority Data
Sep. 14, 2022 (SE) 2230288-9

(51) Int. Cl. A61L 11/00; A61L 9/015; B65F 1/14

(52) U.S. Cl. CPC A61L 11/00; A61L 9/015; B65F 1/14; A61L 2202/11; A61L 2202/14; A61L 2202/15; B65F 2210/128; B65F 2210/129; B65F 2210/172

(57) ABSTRACT

The present invention relates to a waste control unit 1 for generating a flow of gas to affect waste odour in an interior of a waste container 3. The waste control unit 1 comprises a plate element 4 together with wall elements 5a-5d extending around the plate element 4 forming a compartment 6 with an opening 7 arranged opposite the plate element 4 and in which compartment 6 a source of electric power 8 and a gas processor unit 9 are arranged. Further, the waste control unit 1 comprises a gas inlet 10 and a gas outlet 11. The gas inlet 10 is adapted to guide gas from exterior of the waste container 3 to the gas processor unit 9. The gas outlet 11 is adapted to guide gas from the gas processor unit 9 to the interior of the waste container 3. The waste control unit 1 further comprises a digital device 12 adapted to cooperate with the source of electric power 8 and the gas processor unit 9. The compartment 6 is adapted to be arranged with the waste container 3 whereby an outside of the plate element 4 is adapted to face towards the interior of the waste container 3 and the opening 7 of the compartment 6 is adapted to face away from the interior of the waste container 3.

### FIELD OF THE INVENTION

The present invention relates to a waste control unit and a waste container comprising a waste control unit.

### BACKGROUND OF THE INVENTION

Handling of waste, for example household waste, is normally handled such that the waste is thrown into a waste container which is located in vicinity to where the waste is produced. When the waste container is full, the waste is collected by a garbage truck and transported to a waste management facility for further processing. It is common that garbage trucks follow a decided route and a regular schedule to regularly empty the waste containers and collect the waste along the decided route. Collecting and transporting of the waste happens regardless of how much, or how little, waste there is in the waste container, and regardless of what kind of waste that has been thrown into the waste container. Depending on the kind of waste thrown into the waste container, unpleasant odours from the waste may be generated. This may for example be the case if the waste has been thrown into the waste container early in the cycle of collecting the garbage whereby it may take long time until the garbage truck will arrive to empty the waste container. As mentioned, after some time this might lead to unpleasant odour from the waste in the waste container because the driver is following a pre-set schedule and route whereby it may take long time before the waste container may be emptied. Apart from creation of unpleasant odour this further leads to a costly and inefficient process for collecting and processing the waste thrown in the waste container.

### SUMMARY OF THE INVENTION

An object of the present invention is to provide a waste control unit and a waste container comprising a waste control unit to overcome the above mentioned problems.

In relation to mention object, a further object of the invention is to provide a waste control unit which will facilitate assembly and maintenance compared to those of the prior art.

The objects are achieved with a waste control unit according to claim 1. The objects are further achieved with waste container comprising a waste control unit according to claim 16.

The present invention is described as a waste control unit for generating a flow of gas to affect environment in an interior of a waste container. The interior environment in a waste container may relate to odour or bacteria growth which may relate from the garbage thrown into the waste container. The waste control unit comprises a plate element together with wall elements extending around the plate element forming a compartment with an opening arranged opposite the plate element and in which compartment a source of electric power and a gas processor unit are arranged. Further, the waste control unit comprises a gas inlet and a gas outlet. The gas inlet is adapted to guide gas from exterior of the waste container to the gas processor unit. The gas outlet is adapted to guide gas from the gas processor unit to the interior of the waste container. The waste control unit further comprises a digital device adapted to cooperate with the source of electric power and the gas processor unit. The compartment is adapted to be arranged with the waste container whereby an outside of the plate element is adapted to face towards the interior of the waste container and the opening of the compartment is adapted to face away from the interior of the waste container. An effect of the waste control unit is that the components arranged inside the control unit during assembly or service of the control unit are prevented from facing towards the centre of the waste container where the waste is arranged. This has the further effect, especially during assembly or service of the unit, that the risk that components inside the control unit could fall into the waste container thus is reduced.

According to one embodiment, the waste control unit comprises a panel, which panel is arranged to be connected with the waste control unit via connecting elements whereby the panel is arranged in a position closer to the opening than to the plate element. An effect of the panel is to stabilise the control unit and enhance the rigidity of the control unit. A further effect of the panel is to hold and guide elements from exterior of the waste control unit and into the waste control unit.

According to one embodiment, a first wall element of the wall elements is inclined or angled with respect to the plate element, seen from the inside of the compartment, such that angle a between the plate element and the first wall element is greater than 90°. This has the effect that when garbage is thrown into the container garbage and onto the waste control unit it will slide off the surface of the first wall element because it is inclined. Any waste falling on the control unit will thus continue into the inner part of the waste container.

According to one embodiment, a second wall element of the wall elements is inclined or angled with respect to the plate element, seen from the inside of the compartment the angle β between the plate element and the second wall element is greater than 90°. This has the effect that during emptying of the waste container, and the waste container being turned up-side down during the emptying process, any waste which would fall on the waste control unit will thus slide off the surface of the second wall element.

According to one embodiment, the wall elements are defined as areas in one same sheet of material. According to a further embodiment, the wall elements each have a curved arrangement whereby they together form a tubular wall element comprising said wall elements which tubular wall element is a cylindrical shaped tube having an imaginary centre axis extending through the tubular wall element. The effect of the tubular wall element, cylindrical shaped tube, with curved shaped wall elements is that garbage landing on the wall element, because of the curved, inclined, outer surface and because of gravity will slide off the waste control unit and fall to the bottom of the waste container.

According to one embodiment, all wall elements can be sloped to avoid garbage to stay on the compartment walls after the container has been emptied.

According to one embodiment, the gas processor unit comprises a pump and an ozone generator. Ozone has the effect that it may reduce bacterial growth which may generate unpleasant odour. Because ozone has this effect it is useful in eliminating any unpleasant odour which may be created from waste.

According to one embodiment, a gas comprising air enters the gas inlet and is guided via a first guide element to the pump because of suction effect from the pump. The gas inlet is arranged with the panel. The gas inlet communicates with exterior of the waste control unit. The gas inlet also communicates with exterior of the waste container.

According to one embodiment, the air, having passed the pump, enters the ozone generator via an internal guide element between the pump and the ozone generator in which ozone generator the air is processed into a gas comprising ozone which is guided via the gas outlet into the interior of waste container. One effect of the pump is that the pump, when active, sucks air from outside the waste container and guides the air through the pump and into the ozone generator. The generated ozone from the ozone generator enters into the waste container where the ozone will reduce or eliminate odours and bacteria growth which may have been generated or grown from the waste in the waste container. A further effect from the ozone is that it may have a cleaning effect on the inside of the waste container.

According to one embodiment, the gas inlet is arranged with the panel. This has the effect that the gas inlet is stable and has a firm connection to the panel.

According to one embodiment, a second guide element is arranged between the ozone generator and the gas outlet and which second guide element is guided through a lower portion of the waste control unit and adapted to guide gas comprising ozone through the gas outlet into an interior of the waste container. The second guide element has the effect that it can be adjusted to be guided from the ozone generator to different parts of the interior of the waste container. Because the waste control unit is arranged to be positioned in different waste containers and which waste containers may look different with respect to each other, the second guide element is arranged to be adopted to the waste container.

According to one embodiment, the gas outlet is during use of the waste control unit arranged at a distance below the ozone generator whereby the gas comprising ozone and being heavier than air, will flow down from the ozone generator to the gas outlet and exit into the interior of the waste container. This has the effect that no extra pump is needed to move the gas comprising ozone into the interior of the waste container.

According to one embodiment, the source of electric power is in communication with a solar panel. The effect of having a solar panel connected and arranged to provide electric power to the source of electric power is that the waste control unit does not need to be connected to or depend on external power. The source of electric power may for example be a battery. An effect of utilising a battery is that it can prolong operations relating to the handling and processing of the waste, for example that ozone can be generated over a longer time period.

According to one embodiment, the waste control unit comprises a first sensor which is adopted to measure the amount of waste in the waste container and which first sensor is in communication with the digital device. This has the effect that the sensor may generate a signal, or impulse, when the waste container contains a certain amount of waste. The amount, or level, of waste to activate the sensor can be predefined. For example, the waste control unit may be arranged with a light such as a LED-light which light may be arranged to indicate when the waste container is full and needs to be emptied.

According to one embodiment, the waste control unit comprises a second sensor which is adopted to activate upon generation of odour from the waste. Such sensor is a VOC-sensor, also called volatile organic component sensor. The pump and the ozone generator are arranged to communicate with the digital device. The first sensor and the second sensor being the volatile organic component sensor are also arranged to communicate with the digital device. The effect of the volatile organic component sensor is that it can generate a signal when the odour has reached a certain level in the waste container. The generated signal is then communicated to the digital device for processing, whereby the digital device can instruct the pump and the ozone generator when to go into an active as well as a non-active state. This has the effect that the use of power from the power source to the pump and the ozone generator can be optimized since electricity will only be needed when the odour level has reached a certain level in the waste container.

According to one embodiment, the waste control unit comprises a GPS unit, also called a global position system unit. The GPS unit is in communication with the digital device. According to a further embodiment the GPS unit is a GPS tracker. This has the effect that the position and/or the location of the waste container can be identified.

According to one embodiment, the digital device is arranged to communicate with an external communication central unit via an antenna arranged with the waste control unit. The external communication central unit is arranged to receive information from the GPS unit regarding the location of the waste control unit and the first sensor the waste the waste level in the container, from the second sensor the odour level, and information of the power source status. In a further embodiment, the waste control unit may be arranged with a third sensor which is arranged to determine what kind of material the waste consists of, for example if the waste consists of house waste, metal, paper, glass, or other kind of materials. Also, the third sensor may be in communication with the digital device to provide the external communication central unit with the third sensor status. By means of the information sent to the external communication central unit, the route, the time, the frequency, and which waste containers that needs to be emptied can be planned and sent as instructions to the driver of the garbage truck. This planning can be managed via a processor or manually. The external communication central unit can be a mobile device which is arranged in the garbage truck, or a device which is placed elsewhere and thus communicates and sends instructions to the driver of the garbage truck.

According to one embodiment, the panel may be arranged with a display. When a person approaches the waste container provided with the waste control unit the person may on the display receive information or instruction relevant for him or her. Further the panel may comprise a reader functionality which may be arranged for identifying the person who attempts to throw waste in the waste container. For example, when the person has been correctly identified, the waste container which may be arranged with a lock can open the lock allowing the identified person to throw his or hers waste into the waste container. When the person has thrown the waste into the waste container, the waste container may then return to the locked state. The identification of the person may for example performed by utilising a bar code, a QR code or other element which is arranged to cooperate with the reader to identify the person.

According to one embodiment, the lock arranged to lock the lid may be a magnetic lock. The magnetic lock is in communication with the digital device and can be instructed to be locked or unlocked. For example, if wrong waste, wrong material, or dangerous waste which for example may be flammable, has been thrown into the waste container, wherein the lock may be instructed to be arranged in locked status preventing any further material to be thrown into the waste container.

According to one embodiment, the waste container may be arranged with a visible system being in communication with the digital device. The visible system may for example be a lamp having a specific colour which can activate, light up, whereby it will indicate to the driver or personal arriving with the garbage truck which waste container it is he or she shall collect for emptying.

The present invention further refers to a waste container comprising a waste control unit with reference to the mention different embodiments as explained above.

### BRIEF DESCRIPTION OF THE DRAWINGS

The invention is described in more detail below with reference to the appended drawings in which:

FIG. 1 is a view of a waste control unit where a panel is separated from the rest of the waste control unit.

FIG. 2 is a view of the waste control unit in mounted position on part of a waste container.

FIG. 3 is a view of a gas processor unit.

FIG. 4 is a view of a waste control unit where waste control has a cylindrical shape.

FIG. 5 is a view of a system comprising the waste container with the waste control unit being in communication with an external communication central unit.

### DETAILED DESCRIPTION OF PREFERRED EMBODIMENT OF THE INVENTION

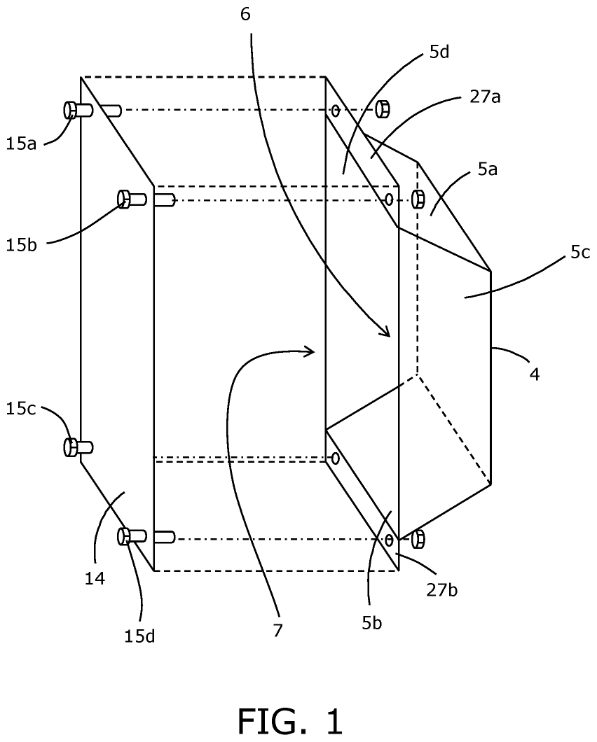

FIG. 1 illustrates a waste control unit 1. The waste control unit 1 comprises wall elements 5a-5d. The wall elements 5a-5d consist of a first wall element 5a, a second wall element 5b, a third wall element 5c, and a fourth wall element 5d. The wall elements 5a-5d are attached with a plate element 4 whereby the wall elements 5a-5d together with the plate element 4 form a compartment 6. The compartment 6 comprises an opening 7 defined by the edges of the wall elements 5a-5d which are opposite their edges which are arranged with the plate element 4. The opening 7 is arranged opposite the plate element 4. A first lip element 27a is arranged to the first wall element 5a. To the second wall element 5b is a second lip element 27b arranged. The waste control unit 1 is further arranged to comprise a panel 14. The panel 14 is arranged to be releasably connected to the compartment 6 by connecting elements 15a-15d which connect the panel 14 to the respective lip element 27a, 27b. The connecting elements 15a-15d may be fastening elements, for example screws and bults, which are arranged to be tightened allowing the lip elements 27a, 27b and the panel 14 to be arranged in a close and fixed position with respect to each other.

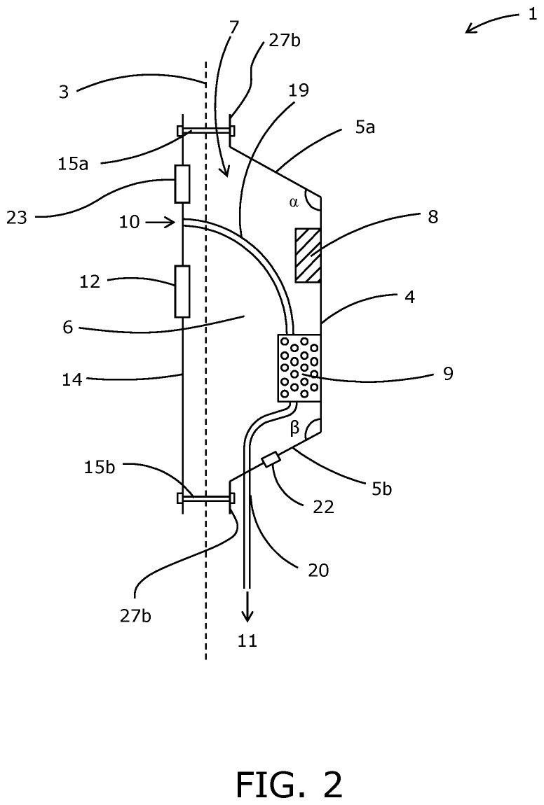

FIG. 2 illustrates a waste control unit 1 in a cross section view. The first wall element 5a is connected to the plate element 4 whereby their respective inside facing towards the compartment 6 form between each other an angle a which is greater than 90 degrees. The other side of the first wall element 5a is an outside. This outside of the first wall element 5a is sloped, or inclined, from the first lip element 27a down to the plate element 4. The slope of the outside of the first wall element 5a admits waste thrown into the waste container 3 and falls onto the first wall element 5a to fall off this surface and continue down and into the waste container 3. The second wall element 5b is connected to the plate element 4 whereby their respective inside facing towards the compartment 6 form between each other an angle β. This angle β may be greater than 90 degrees. Angle a may be equal to angle β.
FIG. 2 further illustrates the compartment 6 which comprises a source of electric power 8, a gas processor unit 9, a first guide element 19, and second guide element 20. The source of electric power 8 may be chargeable battery. The source of electric power 8 and the gas processor unit 9 may be arranged on the inside of the plate element 4. The gas processor unit 9 communicates with the source of electric power 8 (not shown in the figure). The first guide element 19 is with its one end arranged with the gas processor unit 9. The first guide element 19 is with its second end arranged with an opening representing a gas inlet 10 in the panel 14. In one embodiment, the panel 14 is arranged on the outside of the waste container 3 and the waste control unit 1 with lip elements 27a, 27b, wall elements 5a-5d and plate element 4 is arranged on the inside of the waste container 3. In FIG. 2 this part of the waste container 3 is illustrated as the dotted line. The part of the waste container may be prepared, or modified, to contain necessary holes and openings to allow the first guide element 19 to pass through the part of the waste container 3 to the panel 14. The second guide element 20 is with its one end arranged with the gas processor unit 9. The second guide element 20 is arranged to communicate with the interior 2 of the waste container 3.

As illustrated in FIG. 2, the panel 14 is arranged with a digital device 12 and a GPS-unit 23. The digital device 12 is arranged to be in communication with the GPS-unit 23 (not shown in the figure). The digital device 12 is further arranged to be in communication with the source of electrical power 8 (not shown in the figure). Further, the digital device 12 is arranged to be in communication with the gas processor unit 9 (not shown in the figure). In one embodiment of the invention, the digital device 12 or the GPS-unit, or both, may be arranged in the compartment 6 towards one of the inside surfaces of the wall elements 5a-5d or the plate element 4. The waste control unit 1 is held in place and position to the waste container 3 by the connecting elements 15a-15d which may be arranged in the respective corner of the waste control unit 1. The connecting elements 15a-15d are arranged to be placed in through holes or openings in the waste container 3 to secure the waste control unit 1 to the waste container 3.

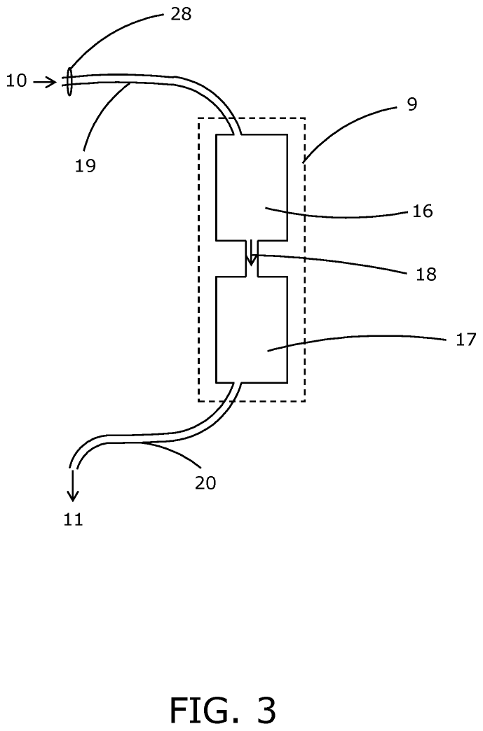

FIG. 3 illustrates the gas processor unit 9. The gas processor unit 9 comprises a pump 16 and an ozone generator 17. Between the pump 16 and the ozone generator 17 an internal guide element 18 is arranged allowing communication therebetween. The pump 16 is arranged with the first guide element 19. Air from outside the waste container 3 enters the gas inlet 10 and into the first guide element 19 and is sucked into the pump 16 when the pump 16 is activated. From the pump 16 the air enters the ozone generator 17 via the internal communication element 18. In the ozone generator 17 the air is processed to produce ozone. The produced ozone exits the ozone generator 18 via the second guide element 20. Ozone enters the interior 2 of the waste container 3 via a gas outlet 11 of the second guide element 20. The gas outlet 11 is arranged to be positioned in the waste container 3 at a lower level than the ozone generator 17. Ozone is heavier than air and will therefor by own force, enter the waste container via the second guide element 20 and the gas outlet 11.

FIG. 3 further illustrates a membrane filter 28 which is arranged with the gas inlet 10 and the first guide element 19. The membrane filter 28 may be arranged on the panel 14 in cooperation with the gas inlet 10. An effect of the membrane filter 28 is that it can prevent passage of liquid, particles, and microorganisms to enter into the first guide element 19, the pump 16 and the ozone generator 17. Because the membrane filter 28 can prevent liquid, particles and microorganisms to reach the gas processor unit 9, formation of nitrous gases or nitric acid in the ozone generator 17 may be avoided or reduced. Nitrous gases or nitric acid in ozone generators may over time have a negative effect on the efficiency how an ozone generator performs whereby it is an aim to try to limit or reduce any such nitrous gases in an ozone generator.

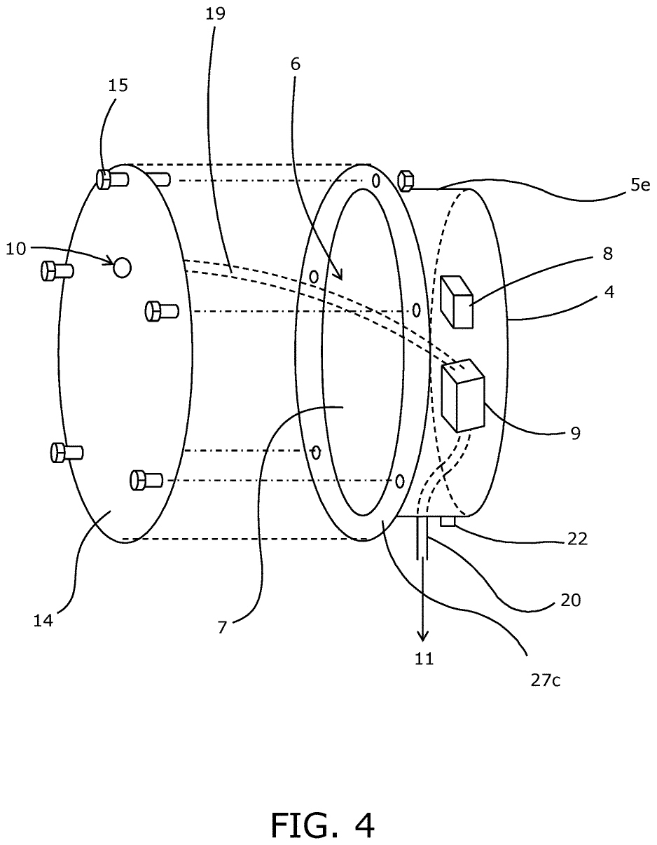

FIG. 4 illustrates an embodiment of the waste control unit 1 comprising a rounded or tubular shaped wall element 5e. The tubular shaped wall element 5e has a cylindric shape comprising one edge that is connected to a plate element 4. Inside the tubular shaped wall element 5e arranged with the plate element 4 a compartment 6 is arranged. Opposite the plate element 4, the tubular shaped wall element 5e an opposite edge is arranged. The opposite edge being circular defines an opening 7 leading into the compartment 6. The tubular shaped wall element 5e with its opposite edge is connected to a lip element 27c. The lip element 27c is a circular and flat element which extends around and is connected to and along the opposite edge of the waste control unit 1. A panel 14 is arranged to be connected to the lip element 27c. The panel 14 may have a circular shape corresponding to an outer periphery of the lip element 27c. The panel 14 is releasably arranged to the lip element 27c by connecting elements 15a-e whereby the panel 14 may be arranged in a fix and secured position to seal the compartment 6 from exterior and to be possible for disassembling for accessing the compartment 6.

During use and when the waste control unit 1 is arranged with the waste container 3, the tubular shaped wall element 5e together with the plate element 4 of the waste control unit 1 is arranged inside the waste container 3. According to one embodiment, the waste control unit 1 may be arranged and positioned through an aperture arranged in the wall of the waste container 3. According to a further embodiment, the waste control unit 1 may be arranged inside the waste container 3 against one of its walls.

FIG. 4 further illustrates the compartment 6 comprising a source of electric power 8, a gas processor unit 9, a first guide element 19, and a second guide element 20. In FIG. 4, the panel 14 is spaced away from the lip element 27c for illustration purpose, therefore the first guide element 19 in the FIG. 4 is dotted to describe its presence in the waste control unit 1. The source of electric power 8 may be a chargeable battery. The source of electric power 8 and the gas processor unit 9 may be arranged on the inside of the plate element 4. The gas processor unit 9 is arranged to communicate with the source of electric power 8 (not shown in the figure). The first guide element 19 with its one end is arranged with the gas processor unit 9. The first guide element 19 with its second end is arranged with an opening representing a gas inlet 10 in the panel 14 for air outside the waste container 3 to be sucked into the first guide element 19. The second guide element 20 is with its one end arranged with the gas processor unit 9. The second guide element 20 is arranged to communicate with the interior 2 of the waste container 3.

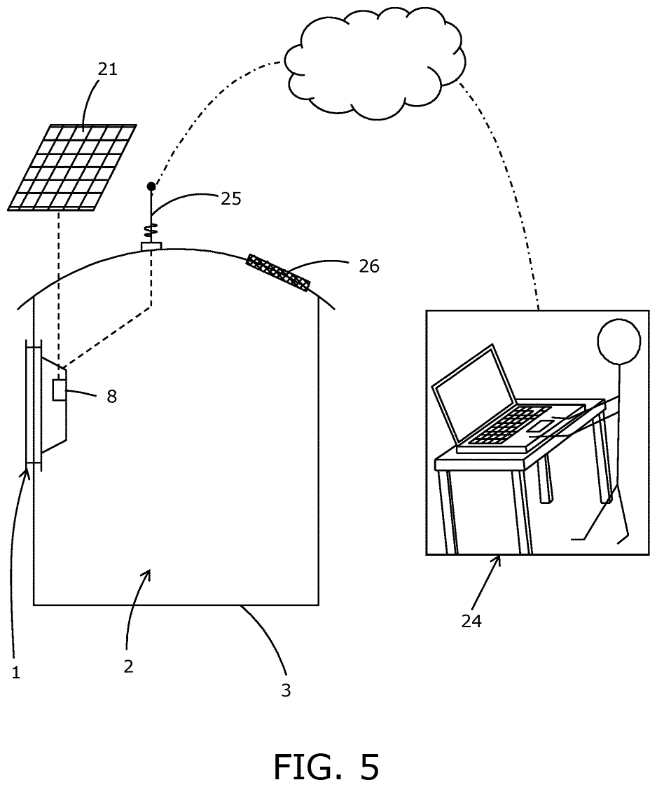

FIG. 5 illustrates the waste container 3 comprising a lid 26 which you can open to throw waste into the waste container 3. The lid 26 may comprise a lock which can be arranged such that it will only open to persons who have the correct key or code to the lock. The lid may further be arranged to be in communication with the digital device 12 (not shown in the figure) whereby the digital device may control and manage the opening of the lid, for example if the waste container 3 is full and is waiting to be emptied then the lid can be set in a close mode.

FIG. 5 further illustrates the waste container 3 comprising the waste control unit 1. The source of electric power 8 is communicating with a solar panel 21. The solar panel 21 is arranged to provide electric energy to the source of electric power 8. The waste control unit 1, via the digital device 12 (not shown in the figure), communicates with an antenna 25. The antenna 25 is arranged on or in direct vicinity to the waste container 3. The digital device 12 is, via the antenna 25, arranged to wireless communicate with an external communication unit 24. The wireless communication may be via internet, cloud solution, blue-tooth, or via other wireless communication media. At the external communication unit 24 an operator will be able to monitor status of the waste container 3 regarding waste level in the waste container 3, the amount of ozone needed to be produced to minimise creation of odour, and the location of the waste container 3. With this data a person at the external communication unit 24 can then optimise when a waste container 3 needs to be emptied as well as optimise the path a driver needs to follow to empty the respective waste containers 3.

Hereinbefore it has been described that the waste control unit may be related to primarily a waste container. It will be appreciated that the principles described above with a waste control unit and system may be applied to other types of waste containers and processes.

### REFERENCE SIGNS

1 waste control unit
2 interior
3 waste container
4 plate element
5a-e wall elements
6 compartment
7 opening
8 source of electric power
9 gas processor unit
10 gas inlet
11 gas outlet
12 digital device
13 outside
14 panel
15a-e connecting elements
α angle α
β angle β
16 pump
17 ozone generator
18 internal guide element
19 first guide element
20 second guide element
21 solar panel
22 first sensor
23 GPS unit
24 external communication central unit
25 antenna
26 lid
27a-c lip element
28 membrane filter

1.-19. (canceled)

20. A waste control unit (1) for generating a flow of gas to affect environment in an interior (2) of a waste container (3), the waste control unit (1) comprises a plate element (4) and wall elements (5a, 5b, 5c, 5d, 5e) extending around the plate element (4) forming a compartment (6) with an opening (7) arranged opposite the plate element (4), in which compartment (6) a source of electric power (8) and a gas processor unit (9) are arranged, which waste control unit (1) comprises a gas inlet (10) and a gas outlet (11), which gas inlet (10) is adapted to guide gas from exterior of the waste container (3) to the gas processor unit (9), which gas outlet (11) is adapted to guide gas from the gas processor unit (9) to the interior (2) of the waste container (3),
which waste control unit (1) comprises a digital device (12) adapted to cooperate with the source of electric power (8) and the gas processor unit (9),
characterised in that,
the compartment (6) is adapted to be arranged with the waste container (3) whereby an outside (13) of the plate element (4) is adapted to face towards the interior (2) of the waste container (3) and the opening (7) of the compartment (6) is adapted to face away from the interior (2) of the waste container (3).

21. A waste control unit (1) according to claim 20, wherein the waste control unit (1) comprises a panel (14) arranged to be connected to the waste control unit (1) via connecting elements (15a, 15b, 15c, 15d), whereby the panel (14) in arranged position is closer to the opening (7) than to the plate element (4).

22. A waste control unit (1) according to claim 20, wherein a first wall element (5a) of the wall elements (15a-d) is sloped or angled with respect to the plate element (4), seen from the inside of the compartment (6), such that angle (a) between the plate element (4) and the first wall element (5a) is greater than 90°.

23. A waste control unit (1) according to claim 20, wherein a second wall element (5b) of the wall elements (15a-d) is sloped or angled with respect to the plate element (4), seen from the inside of the compartment (6), such that angle (P) between the plate element (4) and the second wall element (5a) is greater than 90°.

24. A waste control unit (1) according to claim 20, wherein the respective wall elements (5a-d) are defined as areas in one same sheet of material.

25. A waste control unit (1) according to claim 24, wherein the wall elements (5a-d) each have a curved arrangement whereby they together form a tubular wall element (5e) comprising said wall elements (5a-5d) which tubular wall element (5e) is a cylindrical shaped tube having an imaginary centre axis extending through the tubular wall element (5e).

26. A waste control unit (1) according to claim 20, wherein the gas processor unit (9) comprises a pump (16) and an ozone generator (17).

27. A waste control unit (1) according to claim 26, wherein a gas comprising air enters into the gas inlet (10) and is guided via a first guide element (19) to the pump (16) because of suction effect from the pump (16).

28. A waste control unit (1) according to claim 27, wherein the air having passed the pump (16) thereafter enters the ozone generator (17) via an internal guide element (18) between the pump (16) and the ozone generator (17) in which ozone generator (17) the air is processed into a gas comprising ozone which is guided via the gas outlet (11) into the interior (2) of waste container (3).

29. A waste control unit (1) according to claim 20, wherein the gas inlet (10) is arranged with the panel (14).

30. A waste control unit (1) according to claim 20, wherein a second guide element (20) is arranged between the ozone generator (17) and the gas outlet (11) and which second guide element (20) is guided through a lower portion of the waste control unit (1) and adapted to guide gas comprising ozone through the gas outlet (11) into an interior of the waste container (3).

31. A waste control unit (1) according to claim 30, wherein the second guide element (20) extends from the ozone generator (17) and the waste control unit (1) in a downward direction from the waste control unit (1) and towards an area adopted to contain waste in the waste container (3).

32. A waste control unit (1) according to claim 30, wherein the gas outlet (11), during use of the waste control unit (1) is arranged at a distance below the ozone generator (17) whereby the gas comprising ozone being heavier than air will move by own force down and exit the gas outlet (11).

33. A waste control unit (1) according to claim 20, wherein the source of electric power (8) is in communication with a solar panel (21).

34. A waste control unit (1) according to claim 20, wherein the waste control unit comprises (1) a first sensor (22) which is adopted to measure amount of waste in the waste container (3) and which first sensor (22) is in communication with the digital device (12).

35. A waste control unit (1) according to claim 20, wherein the waste control unit (1) comprises a GPS unit (23) and which GPS unit (23) is in communication with the digital device (12)

36. A waste control unit (1) according to claim 35, wherein the GPS unit (23) is a GPS tracker.

37. A waste control unit (1) according to claim 20, wherein the digital device (12) is arranged to communicate with an external communication central unit via an antenna (25) arranged with the waste control unit (1).

38. A waste container (3) comprising a waste control unit (1) according to claim 20.
